(19) United States
(12) Patent Application Publication
(10) Pub. No.: US 2011/0257687 A1
(43) Pub. Date: Oct. 20, 2011

(54) LOAD SHARING BONE FASTENER AND METHODS OF USE

(75) Inventors: Hai H. Trieu, Cordova, TN (US); Jeff R. Justis, Germantown, TN (US)

(73) Assignee: WARSAW ORTHOPEDIC, INC., Warsaw, IN (US)

(21) Appl. No.: 12/762,421

(22) Filed: Apr. 19, 2010

(51) Int. Cl. A61B 17/70; A61B 17/86

(52) U.S. Cl. 606/267; 606/305

(57) ABSTRACT

A bone fastener includes a first section configured to support a bone construct. A second section is configured for fixation with bone and defines a longitudinal axis. An intermediate flexible section is disposed between the first section and the second section such that the first section is longitudinally aligned with the second section. The intermediate flexible section includes an inner surface that defines a cavity and an open end.

### TECHNICAL FIELD

The present disclosure generally relates to medical devices for the treatment of bone disorders, and more particularly to a flexible bone fastener that is configured to provide load sharing and stabilization in a vertebral rod system.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods may be attached via fasteners to the exterior of two or more vertebral members. This disclosure describes an improvement over these prior art technologies.

### SUMMARY OF THE INVENTION

Accordingly, a flexible bone fastener is disclosed, which is configured to provide load sharing and stabilization in a vertebral rod system.

In one particular embodiment, in accordance with the principles of the present disclosure, a bone fastener is provided. The bone fastener includes a first section configured to support a bone construct. A second section is configured for fixation with bone and defines a longitudinal axis. An intermediate flexible section is disposed between the first section and the second section such that the first section is longitudinally aligned with the second section. The intermediate flexible section includes an inner surface that defines a cavity and an open end.

In one embodiment, the bone fastener includes a head defining a cavity that is configured to support a bone construct, and an elongated bone penetrating member. An arcuate arm extends between the head and the member such that the head is longitudinally aligned with the member. The arm includes an inner surface that defines a cavity and an open end such that the arm is configured to provide damping.

In one embodiment, a vertebral construct is provided. The vertebral construct includes at least two bone fasteners, similar to those described herein, and at least one vertebral rod having a first end and a second end. The first end is supported within a head cavity of a first bone fastener and the second end is supported within a head cavity of a second bone fastener.

### BRIEF DESCRIPTION OF THE DRAWINGS

The present disclosure will become more readily apparent from the specific description accompanied by the following drawings, in which:

FIG. 1 is a side view of one particular embodiment of a bone fastener in accordance with the principles of the present disclosure;

FIG. 2 is a side view of the bone fastener shown in FIG. 1;

FIG. 3 is a side view of one embodiment of the bone fastener shown in FIG. 1;

FIG. 4 is a side view of one embodiment of the bone fastener shown in

FIG. 2;

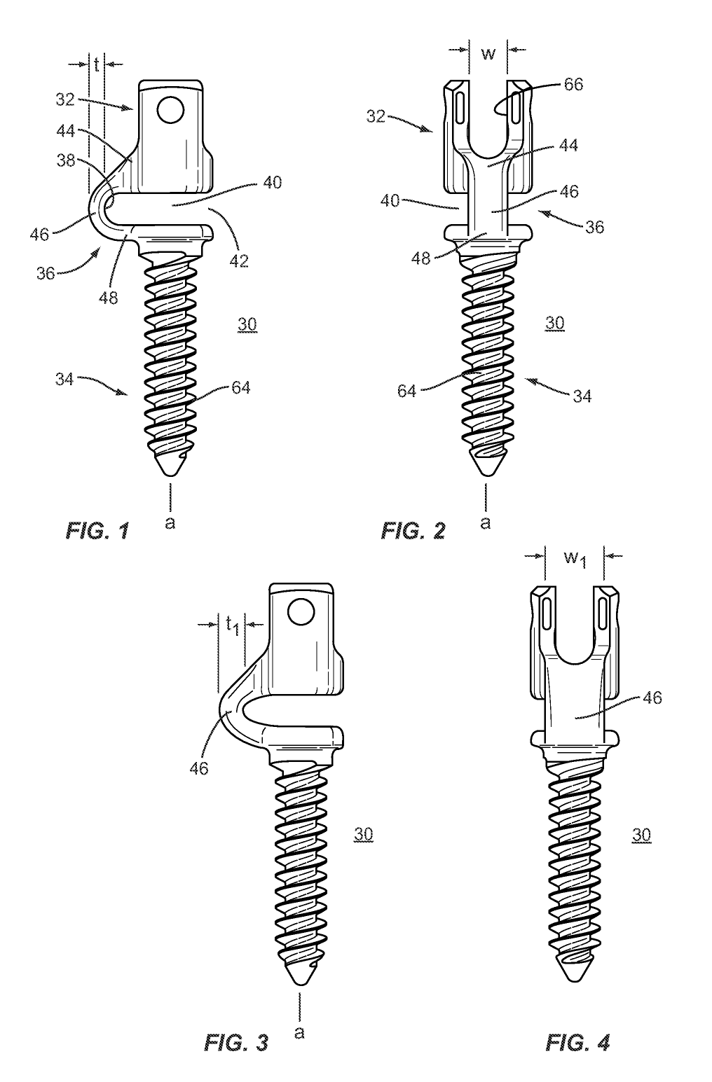

FIG. 5 is a side view of one particular embodiment of a vertebral construct, including the bone fastener shown in FIG. 1, attached to vertebrae in accordance with the principles of the present disclosure;

FIG. 6 is a plan view of the vertebral construct shown in FIG. 5 attached to vertebrae;

FIG. 7 is a side view of one embodiment of the vertebral construct shown in FIG. 5 attached to vertebrae;

FIG. 8 is a side view of one embodiment of the vertebral construct shown in FIG. 5;

FIG. 8A is a side view of one embodiment of the vertebral construct shown in FIG. 5;

FIG. 9 is a side view of one embodiment of the vertebral construct shown in FIG. 8;

FIG. 10 is a side view of one embodiment of the bone fastener shown in FIG. 1;

FIG. 11 is a side view of one embodiment of the bone fastener shown in FIG. 1;

FIG. 12 is a side view of one embodiment of the bone fastener shown in FIG. 1;

FIG. 13 is a side view of one embodiment of the bone fastener shown in FIG. 1;

FIG. 14 is a side view of one embodiment of the bone fastener shown in FIG. 1;

FIG. 15 is a side view of one embodiment of the bone fastener shown in FIG. 1;

FIG. 16 is a side view of one embodiment of the bone fastener shown in FIG. 1;

FIG. 17 is a perspective view of one embodiment of the bone fastener shown in FIG. 1; and

FIG. 18 is a perspective view of one embodiment of the bone fastener shown in FIG. 1.

Like reference numerals indicate similar parts throughout the figures.

### DETAILED DESCRIPTION OF THE INVENTION

The exemplary embodiments of the flexible bone fastener and methods of use disclosed are discussed in terms of medical devices for the treatment of bone disorders and more particularly, in terms of a flexible bone fastener that is configured to provide load sharing and stabilization to a bone construct and adjacent tissues, including bone, for applications such as, for example, a vertebral rod system. It is envisioned that employment of the bone fastener with a vertebral rod system provides stability and maintains structural integrity while reducing stress on spinal elements. The flexible bone fastener may also be used with other constructs such as plates. It is contemplated that a bone construct may include the bone fastener only, in for example fracture repair applications.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft, implantable prosthetics and/or dynamic stabilization applications. It is contemplated that the present disclosure may be employed with other osteal and bone related applications, including those associated with diagnostics and therapeutics. It is further contemplated that the disclosed bone fastener may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The following discussion includes a description of a bone fastener, related components and exemplary methods of employing the bone fastener in accordance with the principles of the present disclosure. Alternate embodiments are also disclosed. Reference will now be made in detail to the exemplary embodiments of the present disclosure, which are illustrated in the accompanying figures. Turning now to FIGS. 1-2, there is illustrated components of a bone fastener 30 in accordance with the principles of the present disclosure.

The components of bone fastener 30 and bone constructs, such as, for example, a vertebral rod system employed therewith, are fabricated from materials suitable for medical applications, including metals, polymers, ceramics, biocompatible materials and/or their composites, depending on the particular application and/or preference of a medical practitioner. For example, bone fastener 30 and/or a vertebral rod 31, discussed below, of the vertebral rod system can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy and silicone. Different components of the vertebral rod system may have alternative material composites to achieve various desired characteristics such as strength, rigidity, elasticity, compliance, biomechanical performance, durability and radiolucency or imaging preference. The components of the vertebral rod system may also be fabricated from a heterogeneous material such as a combination of two or more of the above-described materials.

For example, bone fastener 30 can be formed of two or more materials. In one embodiment, a first section and a second section of bone fastener 30, discussed below, can be fabricated from carbon-reinforced PEEK and an intermediate section can be fabricated from PEEK. In one embodiment, the first and second sections are fabricated from PEEK and the intermediate section is fabricated from carbon-reinforced PEEK. In one embodiment the first and second sections are fabricated from a first material, such as those described above, and the intermediate section is fabricated from a second material such as, for example, Nitinol, PEEK, carbon-PEEK, a titanium alloy and/or a cobalt-chrome alloy. In one embodiment, alternate materials may be employed in a radial direction of bone fastener 30 such that stiff materials such as metals or other composites are used in a core of the fastener sections and an outer sheet of lower modulus polymeric material is used in the outer radial portion of the fastener sections, or vice versa. In one embodiment employing a composite material similar to those described, the first and second sections can have a cylindrical geometry and the intermediate section can have a rectangular or oblong geometry.

As a further example, a resistance member, discussed below, of bone fastener 30 may be fabricated from materials such as silicone, polyurethane, silicone-polyurethane copolymers, polymeric rubbers, polyolefin rubbers, hydrogels, semi-rigid and rigid materials, and biocompatible materials such as elastomers, rubbers, thermoplastic elastomers, thermoset elastomers, elastomeric composites and plastics.

It is envisioned that the components of the vertebral rod system can be manufactured via various methods. For example, bone fastener 30 can be manufactured and assembled via injection-molding, insert-molding, overmolding, compression molding, transfer molding, co-extrusion, pultrusion, dip-coating, spray-coating, powder-coating, porous-coating, machining, milling from a solid stock material, and their combinations. One skilled in the art, however, will realize that such materials and fabrication methods suitable for assembly and manufacture, in accordance with the present disclosure, would be appropriate.

Bone fastener 30 is employed with a vertebral rod system, which is configured for attachment to bone, such as, for example, vertebrae V (as shown, for example, in FIGS. 5-6) during surgical treatment of a spinal disorder, examples of which are discussed herein. Bone fastener 30 includes a first section, such as, for example, a head 32 configured to support a bone construct, such as, for example, vertebral rod 31. A second elongated section, such as, for example, a bone penetrating member 34 defines a longitudinal axis a and is configured for fixation with vertebrae V. Member 34 is threaded for fixation with bone. It is contemplated that member 34 may include alternate bone fixation elements, such as, for example, a nail configuration, barbs, and/or expanding elements.

An intermediate section, such as, for example, an arcuate arm 36 is connected with sections 32, 34 and extends therebetween as a joining section of the components of bone fastener 30. Arm 36 is disposed between head 32 and member 34 such that head 32 is longitudinally aligned with member 34 along longitudinal axis a. Arm 36 includes an inner surface 38 that defines a cavity 40 and an open end 42 such that arm 36 is configured to provide damping. It is envisioned that the components of bone fastener 30 may be monolithically formed, integrally connected or arranged with attaching elements.

Arm 36 is flexible relative to head 32 and member 34, and is configured to provide resistance to movement of head 32 and member 34. Arm 36 has a first end 44, a flexible joint portion 46 and a second end 48. First end 44 and second end 48 are flexible relative to a mid point of flexible joint portion 46. Head 32 is connected with first end 44 and member 34 is connected with second end 48. It is envisioned that arm 36 may provide increasing, variable, constant and/or decreasing resistance.

It is contemplated that sections 32, 34, 36 can be variously dimensioned, for example, with regard to length, width, diameter and thickness. It is further contemplated that the respective cross-sectional geometry or area of sections 32, 34, 36 may have various configurations, for example, round, oval, rectangular, irregular, consistent, variable, uniform and non-uniform. Head 32 may have a different cross-sectional area, geometry, material or material property such as strength, modulus or flexibility relative to member 34.

Arm 36 may have a variable thickness t according to the requirements of the particular application. It is envisioned that thickness t of arm 36 may be in a range of 0.5 millimeters (mm) to 6.0 mm, and preferably 1.0 mm to 4.0 mm. Flexible joint portion 46 has a width w. It is envisioned that width w may be in a range 1 mm to 12 mm, and preferably 3 mm to 8 mm. In one embodiment, flexible joint portion 46 is enlarged relative to head 32 and member 34 such that width w spans a greater distance than the profile of sections 32, 34. This configuration of width w defines a cross-sectional area with thickness t of portion 46.

It is envisioned that arm 36 may have a wide, narrow, round or irregular configuration. It is further envisioned that arm 36 can be variously configured and dimensioned with regard to size, shape, geometry and material. Arm 36 may also have one or a plurality of elements connecting sections 32, 34 such as spaced apart portions, staggered patterns and mesh. Arm 36 may be fabricated from the same or alternative material to sections 32, 34. Arm 36 may also have a different cross-sectional area, geometry or material property such as strength, modulus and flexibility relative to sections 32, 34. Arm 36 may be connected to sections 32, 34 using various methods and structure including molding of a continuous component, mechanical fastening, adhesive bonding and combinations thereof.

It is envisioned that arm 36 has a flexible hinge configuration, which can be offset forward or backward relative to axis a of bone fastener 30 to modify the flexibility or stiffness of the vertebral rod system. It is further envisioned that particular parameters of bone fastener 30 may be selected to modulate the flexibility or stiffness of the vertebral rod system including the material modulus that may correlate to the hardness of a resistance member discussed below, modification of porosity in a range of 50% to 99%, and preferably 75% to 95%, which may include modification of void volume in a range of 50% to 99%, and preferably 75% to 95%. These parameters allow modification of the properties or performance of bone fastener 30 such as strength, durability, flexibility (or stiffness), overall profile and the ability to employ a percutaneous approach, for a particular application.

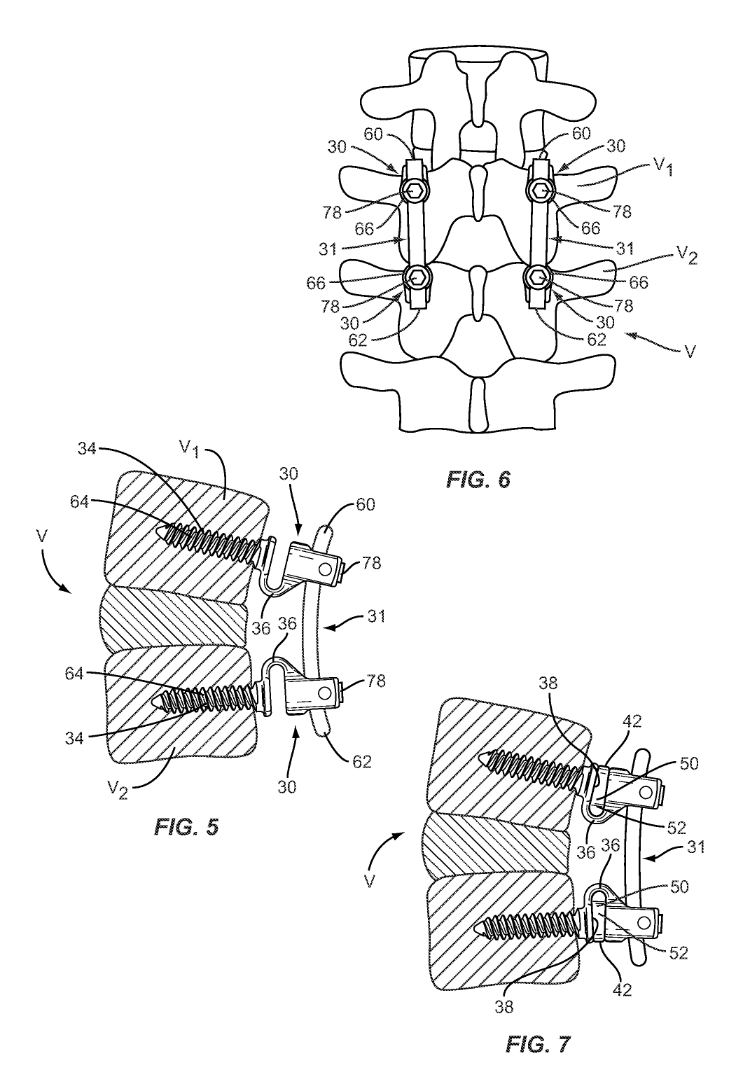

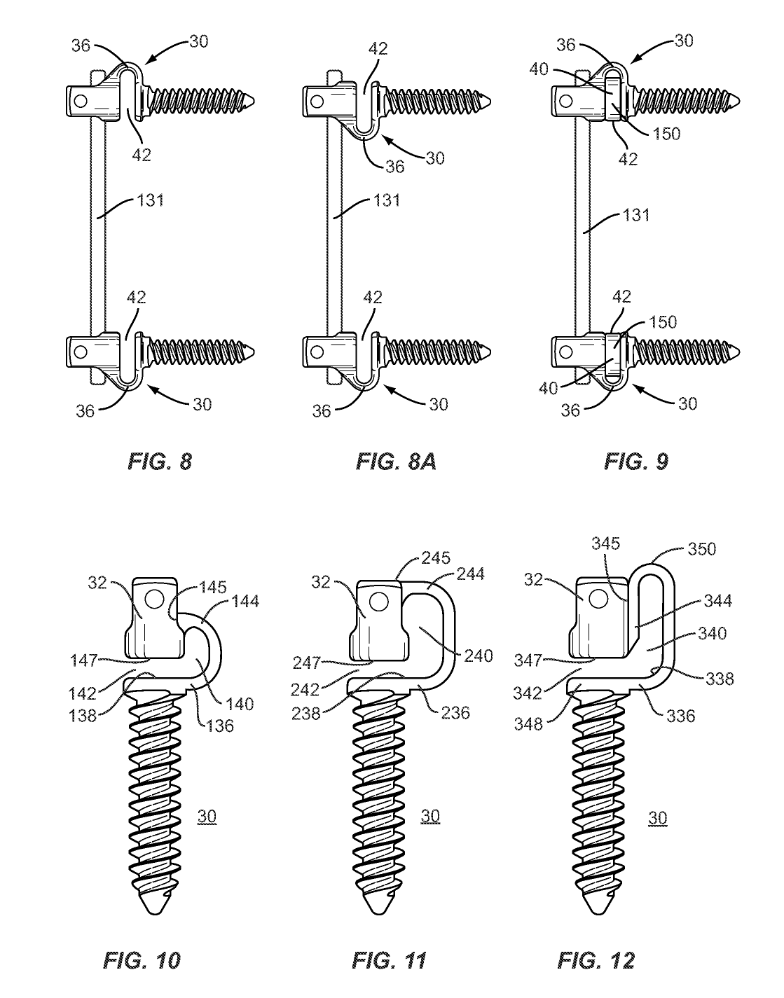

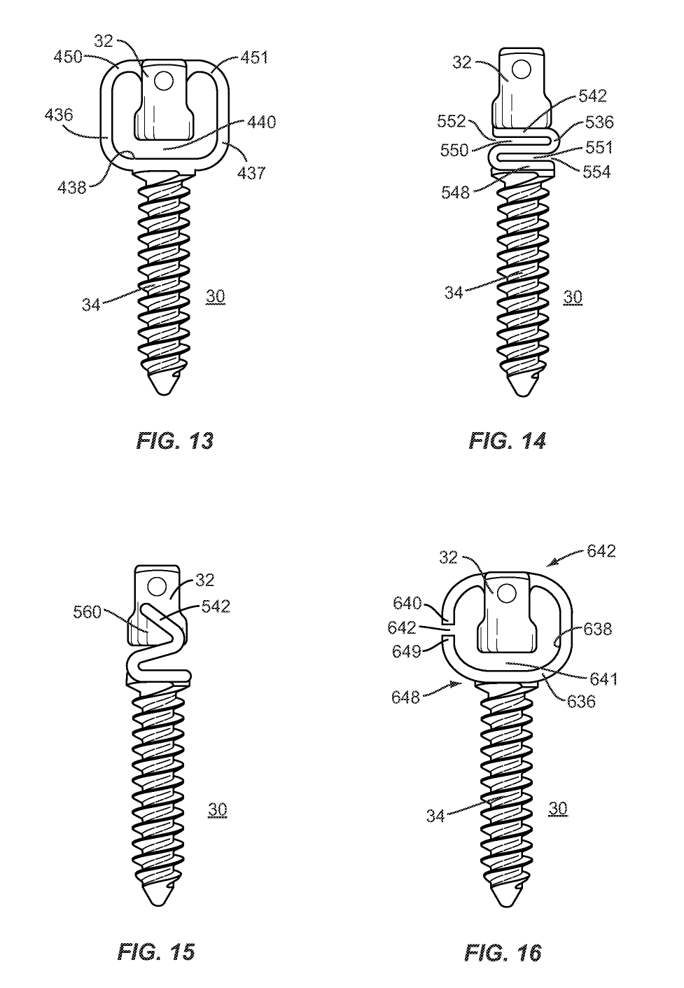

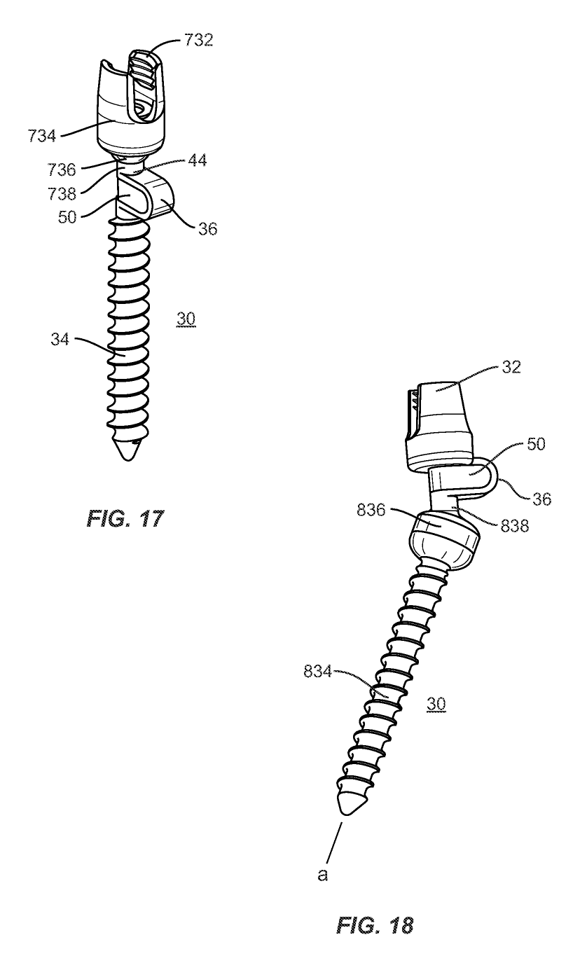

Joint portion 46 has a C-shaped configuration and defines arcuate inner surface 38. It is contemplated that joint portion 46 may have alternative configurations such as S-shaped, U-shaped or W-shaped. In one embodiment, as shown in FIG. 3, joint portion 46 has a V-shape configuration. This configuration has an increased stiffness due to an increased concentration of material at the midpoint of joint portion 46. In one embodiment, as shown in FIG. 4, joint portion 46 has an increased width w₁ to provide increased resistance.

It is contemplated that bone fastener 30 may include one or a plurality of intermediate sections 36 spaced along the length of bone fastener 30. In embodiments including a plurality of sections 36, the multiple sections 36 may be disposed in similar, or alternative orientations such as aligned, non-aligned, offset, facing, opposing, lateral and alternate angular orientation. In one embodiment, joint portion 46 can be bifurcated such that at least a portion thereof can accommodate a vertebral rod.

In a first orientation of bone fastener 30, arm 36 is disposed between head 32 and member 34 such that head 32 is longitudinally aligned with member 34 along longitudinal axis a. It is contemplated that in the first orientation, no flexion, extension or torsional forces are applied to bone fastener 30. As sections 32, 34, 36 move to a second orientation from the first orientation, flexion, extension and/or torsional forces are applied to bone fastener 30. Movement of the components of the vertebral rod system between one or a plurality of orientations is contemplated and may include a range of increasing and decreasing levels of resistance of the components of the vertebral rod system.

In assembly, operation and use, the vertebral rod system including bone fastener 30 is employed with a surgical procedure for treatment of a spinal disorder affecting a section of a spine of a patient, as discussed herein. The bone fastener 30 may also be employed with other surgical procedures. Bone fastener 30 is employed with a surgical procedure for treatment of a condition or injury of an affected section of the spine including vertebrae V, as shown in FIGS. 5-6. It is contemplated that the vertebral rod system including bone fastener 30 is attached to vertebrae V for fusion and/or dynamic stabilization application of the affected section of the spine to facilitate healing and therapeutic treatment, while providing flexion, extension and/or torsion capability. In fusion applications, it is contemplated that bone fastener 30 provides flexibility to a bone construct for improved compliance and less rigidity. In dynamic applications, it is contemplated that bone fastener 30 provides flexibility to the fastener and vertebral construct.

In use, to treat the affected section of the spine, a medical practitioner obtains access to a surgical site including vertebra V in any appropriate manner, such as through incision and retraction of tissues. It is envisioned that the vertebral rod system including bone fastener 30 may be used in any existing surgical method or technique including open surgery, mini-open surgery, minimally invasive surgery and percutaneous surgical implantation, whereby the vertebrae V is accessed through a micro-incision, or sleeve that provides a protected passageway to the area. Once access to the surgical site is obtained, the particular surgical procedure is performed for treating the spinal disorder. The vertebral rod system including bone fastener 30 is then employed to augment the surgical treatment. The vertebral rod system including bone fastener 30 can be delivered or implanted as a pre-assembled device or can be assembled in situ. The vertebral rod system may be completely or partially revised, removed or replaced, for example, replacing rod 31 and/or one or all of the components of bone fastener 30.

Vertebral rod 31 has a rigid, arcuate configuration. A first bone fastener 30 is configured to attach an upper section 60 of vertebral rod 31 to vertebra V₁. A second bone fastener 30 is configured to attach a lower section 62 of vertebral rod 31 to adjacent vertebra V₂. Pilot holes are made in vertebrae V₁, V₂ for receiving first and second bone fasteners 30. Each penetrating member 34 of first and second bone fasteners 30 include a threaded bone engaging portion 64 that are inserted or otherwise connected to vertebrae V₁, V₂, according to the particular requirements of the surgical treatment. Each head 32 of first and second bone fasteners 30 include a bore, or through opening 66 configured to support rod 31, and a set screw 78, which is torqued on to sections 60, 62 to attach rod 31 in place with vertebrae V, as will be described. It is envisioned that vertebral rod 31 may have a semi-rigid or flexible configuration.

As shown in FIG. 6, the vertebral rod system includes two axially aligned and spaced rods 31, with sections 60, 62 extending through bores 66 of heads 32. Set screws 78 of each head 32 are torqued on the end portions of rods 31 to securely attach rods 31 with vertebrae V₁, V₂. Open ends 42 of each bone fastener 30 are facing away from each other. Upon fixation of the vertebral rod system with vertebrae V, bone fasteners 30 each are configured to provide increasing resistance to movement of sections 32, 34 during flexion, extension and/or torsion of the spine. For example, in an unloaded state of bone fasteners 30 and vertebral rods 31, which corresponds to the first orientation of bone fastener 30 discussed above, there are no appreciable tensile, compressive or torsion loads on vertebrae V₁, V₂. In flexion, extension and/or torsion of vertebrae V caused by corresponding movement of the patient, bone fasteners 30 react with increasing resistance facilitating flexibility of rods 31 to a second, third or more orientation(s).

During movement of vertebrae V, for example, in flexion, extension and/or torsion, head 32 moves relative to member 34 to facilitate relative flexibility and/or movement of rod 31 and/or other components of the vertebral rod system. First end 44 and second end 48 can flexibly expand, compress and/or rotate in torsion relative to joint portion 46 such that arm 36 expands and compresses cavity 40. Open end 42 can also expand and compress. This configuration increases resistance during expansion, compression and/or rotation of arm 36. The increase of resistance during flexion, extension and/or torsion provides limited movement of vertebrae V for load sharing and/or dynamic stabilization of the treated area of the spine.

In one embodiment, cavity 40 is configured for disposal of a resistance member, such as, for example, a bumper 50, as shown in FIG. 7. Bumper 50 has an exterior surface 52 that may include a locking part for fixation with a corresponding locking part of arm 36. It is envisioned that bumper 50 may be monolithically formed, integrally connected, employ fastening elements and/or adhesives for disposal with arm 36.

Bumper 50 is elastic and configured to provide variable resistance to movement of sections 32, 34 and 36. It is contemplated that bumper 50 can provide increasing, variable, constant and/or decreasing resistance. Bumper 50 is disposed within cavity 40 and engages surface 38 in a close fitting engagement. Bumper 50 can be variously configured with regard to size, shape, for example, round, oblong, rectangular, triangular, spherical, and irregular shapes. It is envisioned that bumper 50 has a hardness in the range of 30 Shore A to 55 Shore D. The material of bumper 50 can be solid or porous, homogeneous or heterogeneous, single polymer or a blend/composite of more than one polymer. It is contemplated that the resiliency of bumper 50 can prevent creep and improve shape recovery of the vertebral rod system. It is envisioned that bumper 50 is configured to prevent and/or resist closing of open end 42. It is further envisioned that bumper 50 is secured in place with arm 36, and desirably mechanically secured therewith in a configuration to present migration and expulsion therefrom. In other embodiments, bumper 50 can be textured, encapsulated, adhesively bonded and/or over molded with bone fastener 30. Bumper 50 can be inserted with cavity 40 for assembly, or formed in situ by, for example, a pouch, bag or balloon with the bumper configuration being inserted into cavity 40 and injected with a curable material.

During movement of rods 31, for example, in flexion, extension and/or torsion, head 32 moves relative to member 34. First end 44 and second end 48 can flexibly expand, compress and/or rotate in torsion relative to joint portion 46 such that arm 36 expands and compresses cavity 40. Joint portion 46 can flexibly expand circumferentially about bumper 50 such that arm 36 compresses bumper 50; or joint portion 46 can flexibly compress circumferentially about bumper 50. This configuration increases resistance during expansion and compression of arm 36. The increase of resistance during flexion, extension and/or torsion provides limited movement of vertebrae V for load sharing and/or dynamic stabilization of the treated area of the spine.

Bone fastener 30 may be employed as a bone screw, pedicle screw or multi-axial screw (MAS) used in spinal surgery. It is contemplated that bone fastener 30 may be coated with an osteoconductive material such as hydroxyapatite and/or osteoinductive agent such as a bone morphogenic protein for enhanced bony fixation to facilitate motion of the treated spinal area. Bone fastener 30 and bumper 50 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of rod 30 and/or along the length thereof adjacent joint portion 46 or with bumper 50.

Open ends 42 of each bone fastener 30 are facing away from each other. In one embodiment, as shown in FIG. 8, the vertebral rod system includes bone fasteners 30, described above with regard to FIG. 5, and a substantially rigid, linear vertebral rod 131. Arms 36 of each bone fastener 30 are disposed in opposing relation such that open ends 42 are facing each other. In one embodiment, as shown in FIG. 8A, the vertebral rod system includes bone fasteners 30 and rod 131. Arms 36 of each bone fastener 30 are disposed in the same orientation and open ends 42 are facing in the same direction such that open end 42 of a lower arm 36 faces a closed end of an upper arm 36. In one embodiment, as shown in FIG. 9, the vertebral rod system includes bone fastener 30 having a bumper 150, similar to the bone fastener 30 and bumper 50 described with regard to FIG. 7, and substantially rigid, linear vertebral rod 131. Bumper 150 has a circular disc configuration and is disposed within cavity 40. Arms 36 of each bone fastener 30 are disposed in opposing relation such that open ends 42 are facing each other.

Data obtained via computational modeling for the vertebral rod system illustrates calculated deflection of bone fastener 30 under flexion, extension and torsion stress for various embodiments in accordance with the present disclosure, as discussed below. In one example of bone fastener 30, similar to that described with regard to FIGS. 1-2, intermediate section 36 has the dimensional parameters, w=5 mm, t=2 mm, open end 42=4 mm. In flexion, under a 2.0 Newton meter (Nm) bending moment, arm 46 undergoes a stress of 807 megapascals (Mpa) and open end expands a total deformation of 1.46 mm. In extension, under a 2.0 Nm bending moment, arm 46 undergoes a stress of 808 Mpa and open end converges a total deformation of 1.45 mm. In torsion, under a 2.0 Nm torsion moment, arm 46 undergoes a stress of 685 Mpa and open end 42 offsets from longitudinal axis a, a total deformation of 0.46 mm.

In one example of bone fastener 30, similar to that described with regard to FIG. 3, intermediate section 36 has the dimensional parameters, w=5 mm, t=3 mm, open end 42=4 mm. In flexion, under a 2.0 Nm bending moment, arm 46 undergoes a stress of 580 Mpa and open end expands a total deformation of 0.46 mm. In extension, under a 2.0 Nm bending moment, arm 46 undergoes a stress of 601 Mpa and open end converges a total deformation of 0.46 mm. In torsion, under a 2.0 Nm torsion moment, arm 46 undergoes a stress of 387 Mpa and open end 42 offsets from longitudinal axis a, a total deformation of 0.19 mm.

In one example of bone fastener 30, similar to that described with regard to FIG. 4, intermediate section 36 has the dimensional parameters, w=7.5 mm, t=2 mm, open end 42=4 mm. In flexion, under a 2.0 Nm bending moment, arm 46 undergoes a stress of 579 Mpa and open end 42 expands a total deformation of 0.94 mm. In extension, under a 2.0 Nm bending moment, arm 46 undergoes a stress of 587 Mpa and open end 42 converges a total deformation of 0.94 mm. In torsion, under a 2.0 Nm torsion moment, arm 46 undergoes a stress of 380 Mpa and open end 42 offsets from longitudinal axis a, a total deformation of 0.23 mm.

In one example of bone fastener 30, similar to that described with regard to FIG. 4, intermediate section 36 has the dimensional parameters, w=7.5 mm, t=1.75 mm, open end 42=4 mm. In flexion, under a 2.0 Nm bending moment, arm 46 undergoes a stress of 725 Mpa and open end 42 expands a total deformation of 1.08 mm. In extension, under a 2.0 Nm bending moment, arm 46 undergoes a stress of 716 Mpa and open end 42 converges a total deformation of 1.07 mm. In torsion, under a 2.0 Nm torsion moment, arm 46 undergoes a stress of 449 Mpa and open end 42 offsets from longitudinal axis a, a total deformation of 0.27 mm.

In one example of bone fastener 30, similar to that described with regard to FIG. 4, intermediate section 36 has the dimensional parameters, w=7.5 mm, t=2.25 mm, open end 42=4 mm. In flexion, under a 2.0 Nm bending moment, arm 46 undergoes a stress of 532 Mpa and open end 42 expands a total deformation of 0.84 mm. In extension, under a 2.0 Nm bending moment, arm 46 undergoes a stress of 533 Mpa and open end 42 converges a total deformation of 0.84 mm. In torsion, under a 2.0 Nm torsion moment, arm 46 undergoes a stress of 317 Mpa and open end 42 offsets from longitudinal axis a, a total deformation of 0.2 mm.

Referring to FIGS. 10-18, embodiments of bone fastener 30 are shown. In one embodiment, as shown in FIG. 10, bone fastener 30 includes an arcuate arm 136, similar to arm 36 described above, having a first end 144 connected to a mid portion 145 of head 32. An outer surface 147 of head 32 and an inner surface 138 of arm 136 define a cavity 140 of arm 136. Head 32 extends into cavity 140 such that an open end 140 has a reduced dimension.

In one embodiment, as shown in FIG. 11, bone fastener 30 includes an arcuate arm 236, similar to arm 36 described above, having a first end 244 connected to an end portion 245 of head 32. An outer surface 247 of head 32 and an inner surface 238 of arm 236 define a cavity 240 of arm 236. Head 32 extends into cavity 240 such that an open end 242 has a reduced dimension.

In one embodiment, as shown in FIG. 12, bone fastener 30 includes an arcuate arm 336, similar to arm 36 described above, having a first end 344 including an outer surface 350 connected to a side surface 345 of head 32. An outer surface 347 of head 32 and an inner surface 338 of arm 336 define an elongated, arcuate cavity 340 of arm 336. Head 32 extends into cavity 340 to define an open end 342 with a second end 348 of arm 336.

In one embodiment, as shown in FIG. 13, bone fastener 30 includes an intermediate section including a first arcuate arm 436 and a second arcuate arm 437, similar to arm 36 described above. Arm 436 has a first end 450 and arm 437 has a first end 451, which are connected to head 32. Arms 436, 437 extend between head 32 and member 34 to define a closed cavity 440. Arms 436, 437 include an inner surface 438 that defines cavity 440.

In one embodiment, as shown in FIG. 14, bone fastener 30 includes an arcuate arm 536, similar to arm 36 described above, having an undulating configuration. Arm 536 includes a first cavity 550 and an opposing second cavity 551. First cavity 550 has an open end 552 and second cavity 551 has an open end 554. Arm 536 has a first end 542 connected to head 32 and a second end 548 connected to member 34. In one embodiment, as shown in FIG. 15, similar to bone fastener 30 shown in FIG. 14, first end 542 is mounted to a side surface 560 of head 32.

In one embodiment, as shown in FIG. 16, bone fastener 30 includes an arcuate arm 636, similar to arm 36 described above, having an upper portion 642 and a lower portion 648. Head 32 is connected with upper portion 642, in spaced apart relation from a first end 640 of arm 636, and in longitudinal alignment with member 34. A second end 649 of arm 636 is connected to member 34. First end 640 is spaced apart from second end 649 to define an open end 642 of arm 636. Head 32 and an inner surface 638 of arm 636 define a cavity 641.

In one embodiment, as shown in FIG. 17, bone fastener 30 similar to that described above, has a MAS configuration and includes a head 732 having a body 734. Head 732 includes a ball and socket joint 736 that facilitates multi-axial movement of head 732, relative to arm 36 and member 34, including pivot and/or rotation relative to axis a. The intermediate section includes an extension 738 connected to first end 44 such that arm 36 is in spaced apart relation to head 732.

In one embodiment, as shown in FIG. 18, bone fastener 30 similar to that described above, has a MAS configuration and includes a penetrating member 834. Penetrating member 834 includes a ball and socket joint 836 that facilitates multi-axial movement of head 32, relative to member 834, including pivot and/or rotation relative to axis a. Ball and socket joint 836 includes an extension 838 that is connected to arm 36, which includes bumper 50 described with regard to FIG. 7.

It is envisioned that the vertebral rod system described above including bone fastener 30 may be employed with a vertebral rod having an arcuate configuration and an increased length providing the ability to extend over two or more intervertebral elements. It is contemplated that the configuration of the vertebral rod system may provide load sharing, dynamic and/or flexible stabilization over a plurality of intervertebral levels, including treated and untreated vertebral and intervertebral levels.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener comprising:
a first section configured to support a bone construct;
a second section configured for fixation with bone and defining a longitudinal axis; and
an intermediate flexible section being disposed between the first section and the second section such that the first section is longitudinally aligned with the second section, the intermediate flexible section including an inner surface that defines a cavity and an open end.

2. A bone fastener according to claim 1, wherein the intermediate flexible section includes an arcuate arm extending between the first section and the second section.

3. A bone fastener according to claim 2, wherein the arm includes a flexible joint such that the first section and the second section are flexible relative to the joint.

4. A bone fastener according to claim 1, wherein the intermediate flexible section has a first end, a flexible joint and a second end, the first end and the second end being flexible relative to the joint.

5. A bone fastener according to claim 1, wherein the inner surface has an arcuate configuration.

6. A bone fastener according to claim 1, wherein the cavity is configured for disposal of a resistance member.

7. A bone fastener according to claim 6, wherein the resistance member is flexible and configured to provide damping.

8. A bone fastener according to claim 1, wherein the cavity is configured for disposal of a resistance member and the intermediate flexible section has a C-shaped configuration, the resistance member being configured to prevent closing of the open end.

9. A bone fastener according to claim 1, wherein the intermediate flexible section has a first arm and a second arm, each of the first and second arms extending between the first section and the second section.

10. A bone fastener according to claim 1, wherein at least a portion of the bone fastener is fabricated from a PEEK, carbon composite material.

11. A bone fastener according to claim 1, wherein the second section and the intermediate flexible section are connected with the first section in a configuration such that the second section and the intermediate flexible section are movable in a plurality of axial orientations relative to the first section.

12. A bone fastener according to claim 1, wherein the first section and the intermediate flexible section are connected with the second section in a configuration such that the first section and the intermediate flexible section are movable in a plurality of axial orientations relative to the second section.

13. A bone fastener comprising:
a head defining a cavity being configured to support a bone construct;
an elongated bone penetrating member; and
an arcuate arm extending between the head and the member such that the head is longitudinally aligned with the member, the arm including an inner surface that defines a cavity and an open end, such that the arm is configured to provide damping.

14. A bone fastener according to claim 13, wherein the head and the second end of the arm define the open end.

15. A bone fastener according to claim 13, further comprising a resistance element disposed within the cavity and engaging the inner surface in a configuration that provides increasing resistance to convergent movement of the arm.

16. A bone fastener according to claim 13, wherein the arm includes an elastic bumper disposed within the cavity.

17. A bone fastener according to claim 16, wherein the arm has a C-shaped configuration and the elastic bumper is configured to prevent closing of the open end.

18. A vertebral construct comprising:
at least two bone fasteners, each bone fastener including:
a head defining a cavity configured to support a vertebral rod;
an elongated bone penetrating member;
an arcuate arm extending between the head and the member such that the head is longitudinally aligned with a member, the arm including an inner surface that defines a cavity and an open end such that the arm is configured to provide damping; and
at least one vertebral rod having a first end and a second end, the first end being supported within the head cavity of a first bone fastener and the second end being supported within the head cavity of a second bone fastener.

19. A vertebral construct according to claim 18, wherein the open end of a first bone fastener is disposed in facing relation to the open end of a second bone fastener.

20. A vertebral construct according to claim 18, wherein the open end of a first bone fastener is disposed in opposing relation to the open end of a second bone fastener.
